(19) United States
(12) Patent Application Publication
(10) Pub. No.: US 2022/0047293 A1
(43) Pub. Date: Feb. 17, 2022

(54) SURGICAL TOOL AND METHOD OF USE FOR CARPEL TUNNEL RELEASE PROCEDURE

(71) Applicant: Farzad Sadlouparizi, Coquitlam (CA)

(72) Inventor: Farzad Sadlouparizi, Coquitlam (CA)

(21) Appl. No.: 17/399,515

(22) Filed: Aug. 11, 2021

(30) Foreign Application Priority Data
Aug. 17, 2020 (CA) 3090169

(51) Int. Cl. A61B 17/32; A61B 90/00; A61B 17/3211

(52) U.S. Cl. CPC A61B 17/320036; A61B 90/36; A61B 2017/00367; A61B 2017/32113; A61B 17/3211

(57) ABSTRACT

A carpal tunnel release surgical tool has an elongate main body supporting a blade at one end thereof for insertion through a wrist incision. The blade moves between a working position protruding from the main body for cutting the transverse carpal ligament, and a stored position in which the blade is retracted to shield the cutting edge. A light source on the body in proximity to the second end thereof emits light for illuminating the ligament to be cut while the tool is inserted in the body of the patient. An actuator member is supported on the body in operative connection to the blade for actuation by an operator to displace the blade between the working position and the stored position of the blade while the bladed end of the body remains inserted to allow removal of the tool after cutting the ligament.

This application claims foreign priority benefits from Canadian Patent Application 3,090,169, filed Aug. 17, 2020.

### FIELD OF THE INVENTION

The present invention relates to a surgical tool having a retractable blade and which is suitable for insertion through an incision in the wrist of a patient for cutting the transverse carpal ligament of the patient during a carpal tunnel release procedure.

### BACKGROUND

Carpal tunnel syndrome (CTS) is a common nerve entrapment disorder. The symptoms of carpal tunnel syndrome consist of, tingling, numbness, and occasionally pain in the hand. The symptoms are often worse at night but can also be present in the daytime in the worker with a provocative job. Symptoms are often worse with driving or holding a telephone, newspaper, or book.

Appropriate treatment can prevent the progression of this disorder and avoid the development of permanent disability.

In managing the condition, therapy consist of surgery and non-surgery (conservative therapy).

Nonsurgical therapy (Conservative therapy) consist of nocturnal wrist splinting in the neutral position or a glucocorticoid injection, Oral glucocorticoid, physical and occupational therapy techniques, and electrical, magnetic, and laser therapy.

Surgical therapy consists of open surgery and endoscopic surgery.

The approach to the management of patients diagnosed with CTS is based upon the acuity and severity of clinical symptoms and the degree of neurogenic injury as determined by electrodiagnostic studies.

In general, splinting, glucocorticoid injections, and oral glucocorticoids are useful for symptom relief of CTS, but surgery is the treatment of choice for patients with evidence of ongoing nerve damage in the lack of a reversible cause.

For patients with mild to moderate CTS, the rate of successful outcomes with nonsurgical therapy ranges from 20 to 93 percent (1,2).

As an initial nonsurgical therapy, surgical decompression is advised for most patients with CTS who have severe median nerve injury, characterized by significant axonal degeneration on nerve conduction studies (NCS) or denervation on needle EMG reinnervated motor units and fibrillation potentials). Those who lack evidence of significant axonal loss or denervation can be managed initially with nonsurgical measures.

For patients with a clinical diagnosis of CTS who have not had electrodiagnostic studies, initial nonsurgical therapy is reasonable if clinical symptoms are mild. However, those with moderate to severe clinical symptoms who are candidates for surgery should have electrodiagnostic studies first to determine if there is significant axonal loss that might prompt a surgical referral, or if another etiology for median nerve damage can be determined. Patients lacking the electrodiagnostic features of axon loss can be treated initially with nonsurgical measures, even in the presence of severe clinical symptoms.

In the event of failure of initial nonsurgical therapy, although evidence is limited, combined treatment employing splinting with glucocorticoid injection(s), oral glucocorticoids, or other nonsurgical interventions may be more effective than the use of any single modality, as supported in (i) McClure P. Evidence-based practice: an example related to the use of splinting in a patient with carpal tunnel syndrome. J Hand Ther 2003; 16:256, and (ii) Muller M, Tsui D, Schnurr R, et al. Effectiveness of hand therapy interventions in primary management of carpal tunnel syndrome: a systematic review. J Hand Ther 2004; 17:210.

For patients who choose initial nocturnal splinting but remain symptomatic at one month, suggesting continuation of splinting for another one to two months while adding a different nonsurgical modality for CTS rather than stopping splinting. A single injection of methylprednisolone was recommended as the next therapeutic option. Some factors Predict failure of conservative/nonsurgical therapy including: Long duration of symptoms (>6 to 12 months), age greater than 50 years, constant paresthesia, impaired two-point discrimination (>6 mm), positive Phalen sign <30 seconds, prolonged motor and sensory latencies determined by electrodiagnostic testing as supported in (i) Kaplan S J, Glickel S Z, Eaton R G. Predictive factors in the non-surgical treatment of carpal tunnel syndrome. J Hand Surg Br 1990; 15:106, (ii) Stahl S, Yarnitsky D, Volpin G, Fried A. [Conservative therapy in carpal tunnel syndrome]. Harefuah 1996; 130:241, and (iii) Jerosch-Herold C, Shepstone L, Houghton J, et al. Prognostic factors for response to treatment by corticosteroid injection or surgery in carpal tunnel syndrome (palms study): A prospective multicenter cohort study. Muscle Nerve 2019; 60:32.

In comparing surgical decompression with nonsurgical therapy, the most rigorous controlled trial enrolled 116 patients who were randomly assigned to carpal tunnel surgery (open or endoscopic at the surgeon's discretion) or to nonsurgical treatment, as supported in Jarvik J G, Comstock B A, Kliot M, et al. Surgery versus non-surgical therapy for carpal tunnel syndrome: a randomised parallel-group trial. Lancet 2009; 374:1074.

On intention-to-treat analysis at 12 months, both groups showed improvement, but patients assigned to surgery had a significantly better mean functional score on the Carpal Tunnel Syndrome Assessment Questionnaire than those assigned to nonsurgical therapy (−0.4, 95% CI 0.11-0.70) as supported in Jarvik J G, Comstock B A, Kliot M, et al. Surgery versus non-surgical therapy for carpal tunnel syndrome: a randomised parallel-group trial. Lancet 2009; 374:1074.

Surgical treatment of CTS appears to be more effective than splinting, although the evidence is limited to a few relatively low-quality trials, as supported in Verdugo R J, Salinas R A, Castillo J L, Cea J G. Surgical versus non-surgical treatment for carpal tunnel syndrome. Cochrane Database Syst Rev 2008; :CD001552.

Surgery is more effective for sustained relief of symptoms from CTS than local glucocorticoid injection. However, short-term results of glucocorticoid injection may be better than those following carpal tunnel release surgery, the advantage is lost over the course of one year following the procedure.

Surgical treatment may involve open or endoscopic technique.

The goal of either approach is to decrease pressure upon the median nerve at the wrist by dividing the transverse carpal ligament and antebrachial fascia.

Indications for surgery include persistent numbness and pain, motor dysfunction with diminished grip or pinch grasping.

Indication for surgery include mild CTS unresponsive to conservative measures, or moderate-to severe CTS associated with axonal loss or denervation on electrodiagnostic studies.

Surgery consist of two main techniques: 1—The open carpal tunnel release, which can be performed through a standard incision or a limited incision, and 2—Endoscopic carpal tunnel release, which can be done through a single or double portal.

Each procedure has its advantages and disadvantages, and there is controversy among prominent hand surgeons as to the best technique. Surgeon experience and preference is therefore the main determining factor in the technique selection.

The open surgery provides a complete view of the anatomy and possible anomalies, thereby declining the risk of injury to critical structures. This approach also permit exploration of the carpal canal or biopsy of the tenosynovium to rule out systemic inflammatory disease, which may not be possible with the endoscopic technique.

Regarding endoscopic technique, Due to preservation of the palmar fascia, subcutaneous fat, and skin, endoscopic median nerve decompression may cause less scar tenderness and an earlier return to work compared with the open surgery. Although, good visualization is essential for the endoscopic technique. If this cannot be achieved, one must change to the open technique.

The incidence of long-term disability related to complications from carpal tunnel release surgery should not exceed 1 to 2 percent as supported in Bland J D. Treatment of carpal tunnel syndrome. Muscle Nerve 2007; 36:167.

Complications of surgery for CTS consist of inadequate division of the transverse carpal ligament, Injuries of the recurrent motor and palmar cutaneous branches of the median nerve, lacerations of the median and ulnar trunk, vascular injuries of the superficial palmar arch, painful scar formation, complex regional pain syndrome, postoperative wound infections as supported in Bland J D. Treatment of carpal tunnel syndrome. Muscle Nerve 2007; 36:167. Incomplete release of the transverse carpal ligament is the most frequent complication of surgery for CTS and is usually associated with errors in surgical technique, such as poor choice of incision and inadequate exposure as supported in Bland J D. Treatment of carpal tunnel syndrome. Muscle Nerve 2007; 36:167.

Regarding open compered with endoscopic techniques, the outcomes appear to be equivalent, as supported in (i) Vasiliadis H S, Georgoulas P, Shrier I, et al. Endoscopic release for carpal tunnel syndrome. Cochrane Database Syst Rev 2014; :CD008265, and (ii) Zuo D, Zhou Z, Wang H, et al. Endoscopic versus open carpal tunnel release for idiopathic carpal tunnel syndrome: a meta-analysis of randomized controlled trials. J Orthop Surg Res 2015; 10:12. However, some studies show a more rapid postoperative recovery and earlier return to work with the endoscopic technique as supported in Saw N L, Jones S, Shepstone L, et al. Early outcome and cost-effectiveness of endoscopic versus open carpal tunnel release: a randomized prospective trial. J Hand Surg Br 2003; 28:444, others have found no significant difference for time to return to work between the two techniques as supported in Macdermid J C, Richards R S, Roth J H, et al. Endoscopic versus open carpal tunnel release: a randomized trial. J Hand Surg Am 2003; 28:475.

The endoscopic technique may associate with less postoperative pain and tenderness of the scar as supported in Brown R A, Gelberman R H, Seiler J G 3rd, et al. Carpal tunnel release. A prospective, randomized assessment of open and endoscopic methods. J Bone Joint Surg Am 1993; 75:1265, but the degree of this benefit is modest, as supported in Atroshi I, Larsson G U, Ornstein E, et al. Outcomes of endoscopic surgery compared with open surgery for carpal tunnel syndrome among employed patients: randomised controlled trial. BMJ 2006; 332:1473.

Digital flexor tendon mechanics are closer to normal following endoscopic carpal tunnel release than with the open technique, as supported in Brown R K, Peimer C A. Changes in digital flexor tendon mechanics after endoscopic and open carpal tunnel releases in cadaver wrists. J Hand Surg Am 2000; 25:112.

### SUMMARY OF THE INVENTION

According to one aspect of the invention there is provided a surgical tool comprising:

a main body which is elongated in a longitudinal direction between a first end and a second end of the body;

a blade supported on the main body in proximity to the second end of the main body;

the blade having a cutting edge along one side of the blade;

the blade being movable between a working position in which the cutting edge of the blade protrudes radially outwardly from the main body transversely to the longitudinal direction, and a stored position in which the blade is retracted radially inwardly towards the main body in relation to the working position;

a light source on the body in proximity to the second end of the main body so as to be arranged to emit light from the main body; and

an actuator member supported on the body in operative connection to the blade so as to be actuable by an operator of the tool to displace the blade between the working position and the stored position of the blade.

The surgical tool described herein is a small, disposable knife which passed under the transverse carpal ligament for cutting the ligament without needing expensive endoscopic instruments which are not available in several centers especially in underdeveloped and poor countries, with very small incision on the wrist and decreased compression on the nerve during surgery in comparison to endoscopic carpal tunnel syndrome and needs only one small incision despite two incisions in traditional endoscopic surgery.

The surgical tool is a small disposable knife invented by an expert surgeon. The device is simply used with minimal training required. This device not only decreases invasiveness of traditional open surgery but also eliminates needing complicated and difficult manners in endoscopic surgery.

The blade is preferably pivotally coupled to the main body so as to be pivotal between the working position and the stored position of the blade. The cutting edge of the blade is preferably retracted into a hollow interior of the main body in the stored position of the blade. Preferably the cutting edge of the blade faces towards first end of the main body in the working position. The cutting edge of the blade may follow a concave curvature along a length of the blade. In the illustrated embodiment, a distal end of the blade is folded towards the first end of the main body from the working position to the stored position of the blade such that the cutting edge of the blade is oriented in the longitudinal direction of the main body in the stored position.

A spring may be operatively connected to the blade so as to be arranged to bias the blade from the working position towards the stored position. Preferably the spring is received within a hollow interior of the main body and is coupled a first end to the main body and at a second end to the blade. The spring may extend in the longitudinal direction of the main body under tension in the working position of the blade.

Preferably the actuator member is slidably supported on the main body and pivotally coupled to the blade such that the actuator member slides longitudinally relative to the main body together with pivotal movement of the blade relative to the body between the working and stored positions. The actuator member may comprise a shaft received within a hollow interior of the main body in which the shaft has a first end in proximity to the first end of the main body and a second end pivotally coupled on an actuator lobe of the blade. The actuator lobe and the cutting edge of the blade are preferably diametrically opposite one another as shown in the accompanying figures. The first end of the shaft may protrude from the main body for gripping by the operator.

The light source preferably comprises a light emitting device, for example a light emitting diode (LED), supported on the main body in proximity to the second end of the main body. The tool preferably further comprises a battery supported within a cavity in the main body to provide electrical power to the light emitting device, and an actuator switch on the main body in operative connection with the light source for actuating the light source in which the actuator switch is externally accessible on the main body.

### BRIEF DESCRIPTION OF THE DRAWINGS

One embodiment of the invention will now be described in conjunction with the accompanying drawings in which:

FIG. 1 is an illustration showing an incision location for performing a prior art open carpal tunnel release surgery;

FIG. 2 is an illustration showing tool placement in a prior art endoscopic carpal tunnel release surgery and the resulting cut in the transverse carpal ligament;

FIG. 3 is a perspective view of the surgical tool according to the present invention;

FIG. 4 is a sectional view of the surgical tool along the line 4-4 in FIG. 3, showing the blade in the working position;

FIG. 5 is a sectional view of the surgical tool along the line 4-4 in FIG. 3, showing the blade in the stored position; and

FIG. 6 is a schematic illustration of the surgical tool according to the present invention when used in a carpel tunnel release procedure.

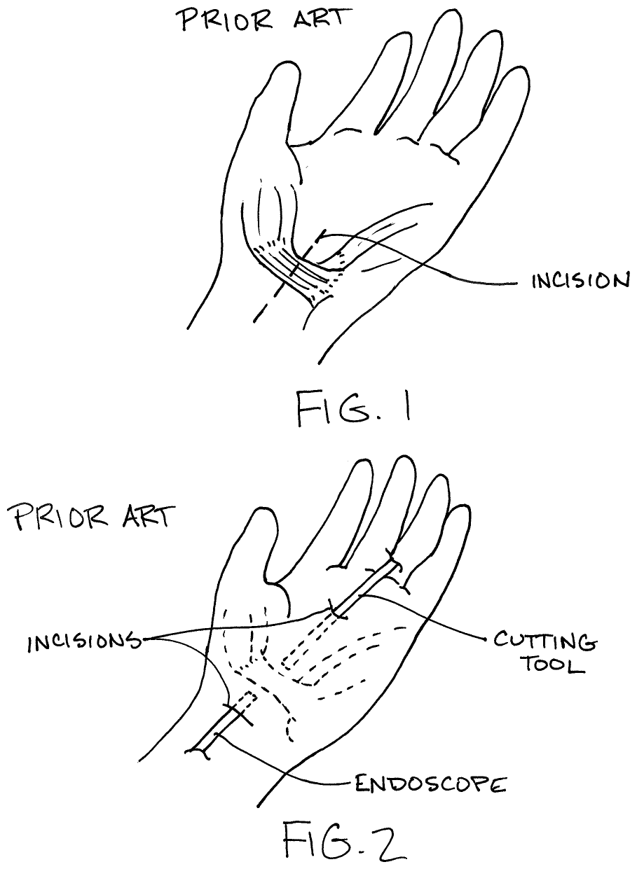

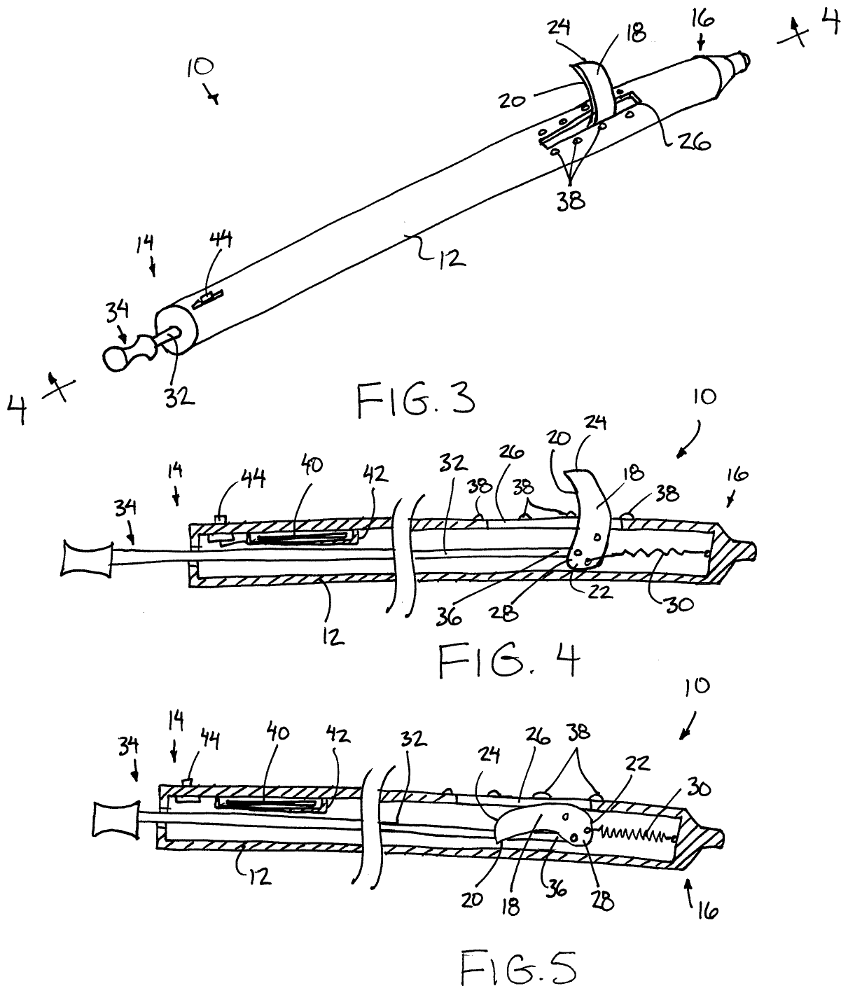

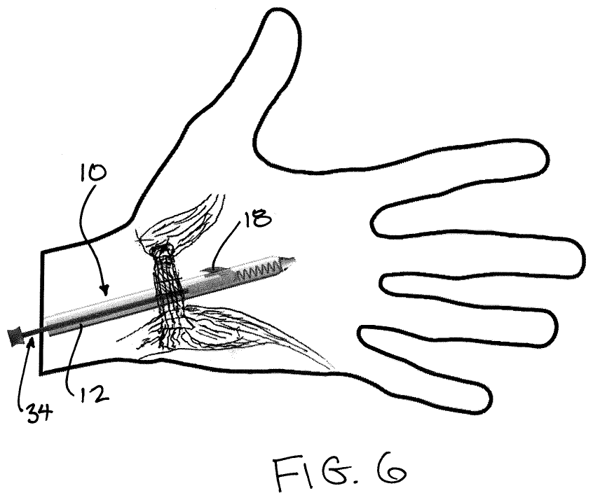

In the drawings like characters of reference indicate corresponding parts in the different figures.

### DETAILED DESCRIPTION

Referring to the accompanying figures, there is illustrated a surgical tool 10 for use in a surgical carpel tunnel release procedure. The surgical tool 10 can replace expensive and time-consuming endoscopic surgery without needing complicated and difficult techniques and tools normally used in endoscopic surgery. The surgical tool 10 described herein is a simple, cheap and very user-friendly device which is decreased invasiveness, complexities and cost compared to traditional carpal tunnel release procedures, including traditional endoscopic and open carpal tunnel surgery.

The tool 10 generally includes a main body 12 which is elongate in a longitudinal direction between a first end 14 and a second end 16 which are opposite one another. The main body is tubular in shape, having a hollow interior passage spanning substantially the full length of the main body between the opposing first and second end thereof. The second end of the body is smooth and tapered in shape for ease of insertion into the body of a patient through a suitable incision as described in further detail below.

A blade 18 is mounted on the main body 12 in proximity to the second end 16 thereof. More particularly the blade comprises a flat rigid plate of material having a sharpened cutting-edge 20 formed along one side thereof. The cutting edge 20 spans substantially the full length of the blade generally in a longitudinal direction of the blade from a first end 22 of the blade that is pivotally coupled to the main body 12 to a second end 24 of the blade which is distal from the pivotal connection. The cutting edge 20 follows a concave curvature while extending in the longitudinal direction between the first and second ends of the blade.

The first end of the blade 18 is received within the hollow interior of the main body 12 in proximity to the second end of the body for pivotal movement about a pivot axis oriented perpendicularly to the longitudinal direction of the main body. The blade 18 generally pivots between a working position protruding outward from the main body for cutting, and a stored position in which the blade is retracted inwardly towards the body relative to the working position.

More particularly, in the working position the cutting edge 20 of the blade is oriented to extend generally radially outwardly, transversely to the longitudinal direction of the body while facing towards the first end of the main body 12. A blade opening 26 is provided in the form of an elongated slot formed along one side of the main body in alignment with the mounting location of the blade 18 to receive the blade protruding therethrough in the working position.

In the stored position, the second end of the blade is displaced radially inwardly and longitudinally toward the first end of the main body as the blade is folded from the working position towards the stored position. In the resulting stored position, at least the cutting edge 20 of the blade and in some instances the entirety of the body of the blade can be retracted into the hollow interior of the main body, such that the cutting edge extends generally in the longitudinal direction of the main body.

The body of the blade 18 further includes an actuating lobe 28 which extends from the pivot axis of the blade diametrically opposite to the cutting edge.

A spring 30 is received within the hollow interior of the main body 12 to extend under tension in the longitudinal direction of the main body between a first end pivotally coupled to the actuating lobe 28 of the blade and a second end coupled to the main body at the second end of the main body. The tension of the spring as a result of the stretching of the spring in the working position acts to pull the actuating lobe towards the second end of the main body which in turn biases the second end 24 of the blade to pivot toward the first end of the main body corresponding to the stored position thereof.

An actuator member 32 is provided on the main body 12 for actuating displacement of the blade between the stored position and the working position thereof. The actuator member 32 comprises an elongated shaft supported within the hollow interior of the main body 12 so as to be longitudinally slidable in the longitudinal direction relative to the main body 12. The shaft of the actuator member 32 extends longitudinally between (i) a first end 34 protruding through an actuator opening at the first end of the main body to extend longitudinally outward beyond the end of the main body and (ii) a second end 36 which is pivotally coupled to the actuating lobe 28 of the blade 18 at a location spaced radially from the pivot axis at a location which is generally diametrically opposite from the second end 24 of the blade in relation to the pivot axis.

The operator of the tool grips the first end 34 of the actuator member 32 and displaces the actuator member longitudinally relative to the main body to pivot the blade between the working and stored positions thereof with longitudinal sliding of the actuator member. Displacing the actuator member longitudinally outward from the main body acts to open the blade 18 towards the working position, whereas pushing the actuator member 32 into the main body acts to close the blade towards the stored position thereof.

A light source is supported on the main body 12 in the form of a plurality of light emitting diodes 38 supported in proximity to the second end of the main body. In particular rows of LED bulbs 38 are supported alongside the slotted blade opening 26 such that the rows of bulbs are oriented in the longitudinal direction. The bulbs are oriented to emit light such that the light is emitted radially outwardly from the main body in the same direction that the blade protrudes in the working position.

A battery 40 is supported within a respective battery cavity 42 within the hollow interior of the body. A suitable circuit connects the battery 40 to the light emitting diodes 38 for providing electrical power to the diodes. An actuation switch 44 forms part of the circuitry between the battery 40 and the diodes 38. The switch 44 is externally accessible on the main body in proximity to the first end thereof so that a user can actuate illumination of the light emitting diodes by actuating the switch.

Use of the tool to perform a carpal tunnel release procedure begins with an operator forming an incision in the wrist area of the patient for insertion of the second end of the main body into the body of the patient. The light emitting diodes 38 are actuated to emit light which is visible through the tissue of the patient as the second end of the tool is positioned in proximity to the transverse carpal ligament of the patient. The illumination by the LEDs 38 assists in guiding position of the tool relative to the transverse carpal ligament. Once the tool has been properly aligned with the ligament, the operator grips the first end of the actuator member 32 to longitudinally slide the actuator member outwardly from the main body sufficiently to displace the blade 18 from the stored position to the working position thereof. While maintaining the blade in the working position, the tool is longitudinally displaced relative to the patient so that the blade cuts through the transverse carpal ligament of the patient. Once the ligament has been cut, the actuator member 32 is pushed inwardly into the main body 12 sufficiently to displace the blade from the working position to the stored position thereof. With the blade remaining in the stored position, the tool is removed from the patient. The incision is subsequently sutured to complete the procedure.

Since various modifications can be made in my invention as herein above described, and many apparently widely different embodiments of same made, it is intended that all matter contained in the accompanying specification shall be interpreted as illustrative only and not in a limiting sense.

1. A surgical tool comprising:
a main body which is elongated in a longitudinal direction between a first end and a second end of the body;
a blade supported on the main body in proximity to the second end of the main body;
the blade having a cutting edge along one side of the blade;
the blade being movable between a working position in which the cutting edge of the blade protrudes radially outwardly from the main body transversely to the longitudinal direction, and a stored position in which the blade is retracted radially inwardly towards the main body in relation to the working position;
a light source on the body in proximity to the second end of the main body so as to be arranged to emit light from the main body; and
an actuator member supported on the body in operative connection to the blade so as to be actuable by an operator of the tool to displace the blade between the working position and the stored position of the blade.

2. The tool according to claim 1 wherein the blade is pivotally coupled to the main body so as to be pivotal between the working position and the stored position of the blade.

3. The tool according to claim 1 wherein the cutting edge of the blade is retracted into a hollow interior of the main body in the stored position of the blade.

4. The tool according to claim 1 wherein the cutting edge of the blade faces towards first end of the main body in the working position.

5. The tool according to claim 1 wherein the cutting edge follows a concave curvature along a length of the blade.

6. The tool according to claim 1 wherein a distal end of the blade is folded towards the first end of the main body from the working position to the stored position of the blade and the cutting edge of the blade is oriented in the longitudinal direction of the main body in the stored position.

7. The tool according to claim 1 further comprising a spring operatively connected to the blade so as to be arranged to bias the blade from the working position towards the stored position.

8. The tool according to claim 7 wherein the spring is received within a hollow interior of the main body and is coupled a first end to the main body and at a second end to the blade.

9. The tool according to claim 7 wherein the spring extends in the longitudinal direction of the main body under tension.

10. The tool according to claim 1 wherein the actuator member is slidably supported on the main body and pivotally coupled to the blade such that the actuator member slides longitudinally relative to the main body together with pivotal movement of the blade relative to the body between the working and stored positions.

11. The tool according to claim 10 wherein the actuator member is a shaft received within a hollow interior of the main body, the shaft having a first end in proximity to the first end of the main body and a second end pivotally coupled on an actuator lobe of the blade, the actuator lobe and the cutting edge of the blade being diametrically opposite one another.

12. The tool according to claim 11 wherein the first end of the shaft protrudes from the main body for gripping by the operator.

13. The tool according to claim 1 wherein the light source comprises a light emitting device supported on the main body in proximity to the second end of the main body.

14. The tool according to claim 13 further comprising a battery supported within a cavity in the main body to provide electrical power to the light emitting device.

15. The tool according to claim 13 further comprising an actuator switch on the main body in operative connection with the light source for actuating the light source, the actuator switch being externally accessible on the main body.
